(19) United States
(12) Patent Application Publication
(10) Pub. No.: US 2026/0207114 A1
(43) Pub. Date: Jul. 23, 2026

(54) SYSTEMS AND METHODS OF DETECTION OF CEREBRAL ROTATION, CUNCUSSIONS, SUB-CONCUSSIONS, AND CTE FUTURE RISK ASSESSMENT AND PREVENTION

(71) Applicants: David YONCE, Malvern, PA (US); Judith MCMINN, London (GB)

(72) Inventors: David YONCE, Malvern, PA (US); Judith MCMINN, London (GB)

(21) Appl. No.: 19/141,117

(22) PCT Filed: Dec. 19, 2023
(86) PCT No.: PCT/US23/84883

§ 371 Date: Jun. 19, 2025

Related U.S. Application Data
(60) Provisional application No. 63/433,577, filed on Dec. 19, 2022.

(51) Int. Cl. A61B 5/369; A61B 5/00; A61B 5/11

(52) U.S. Cl. CPC A61B 5/369; A61B 5/1123; A61B 5/6803

(57) ABSTRACT

A system and method of detecting, monitoring for, and preventing potential brain injury events such as head impacts, concussions, sub-concussions, dangerous head rotation or movement, which may or may not be caused by direct or indirect impacts to a person's head. The system and method include one or more sensors able to detect one or more forces, rotational movements, forces, or accelerations, experienced by a person's head/brain upon an event such as a receiving a rotational force impact. The system and method can monitor these events (e.g., sub-concussions) causing impacts over time and is able to generate or output recommended courses of action in order to limit or prevent debilitating injuries later in life, including CTE.

### PRIORITY

The present application claims the benefit of U.S. Provisional Patent Application No. 63/433,577, filed on Dec. 19, 2022, the entirety of which is incorporated herein by reference.

### FIELD

The present invention relates generally to electroencephalogram (“EEG”) systems, architectures, and methods related to detecting, measuring, and monitoring subjects, and, more particularly, to generally flexible biosensors, EEG systems, architectures, electrodes, and methods of use and treatment related to concussions, sub-concussions and potential future health risks from Chronic Traumatic Encephalopathy (“CTE”).

### BACKGROUND

The awareness of the dangers of head impacts from contact sporting events has grown considerably over the past decades. It has been known for decades that head injuries can have a significant impact on an individual's cognitive function, psychiatric and mental health. In fact, in the early 1920's, the Journal of the American Medical Association coined the term “Punch Drunk Syndrome” after noticing that career boxers were experience similar symptoms, including tremors, slowed movement, speech problems and confusion. Eventually, the medical community made a connection between the “Punch Drunk” symptoms and concussions.

It was not until the early 2000's, however, that the medical community identified a unique, longer-term brain injury called Chronic Traumatic Encephalopathy (“CTE”). CTE creates symptoms of early onset dementia, and in many ways mirrors the brain degradation brought on by Alzheimer's Disease. Thought to be associated with an athlete receiving multiple concussions, CTE heightened the attention or focus on concussions, protective headgear, and overall minimizing the acute burden from a head injury.

Today, there are concussion protocols in place for many major sporting leagues. These protocols require athletes to complete a subjective concussion baseline screening/testing using a couple of commercially available devices to help identify the presence of a concussion. The baseline reading is compared to one or more readings obtained immediately after a concussive event, or in one or more follow-on periods from the concussive event.

In the last several years, however, scientists have begun to discover that the link between acute concussions and long-term CTE is not as strong as initially believed. There are now many examples of athletes receiving multiple concussions and not developing CTE. Interestingly, the converse is also true with up to 20% of people with CTE having never had a reported concussion. These data points suggest that only monitoring a subject or player for concussions does not accurately predict a risk of CTE, or that efforts to reduce a concussion will reduce the risk factor of CTE in life or in sports.

Scientists now believe that CTE is likely caused by repetitive impacts to a user's head. These repetitive impacts may cause sub-concussive impacts (i.e., impacts that do not produce overt signs or symptoms commonly associated with concussions) that cause damage to the brain when experienced repeatedly. Recent research demonstrates that that these repeated impacts and sub-concussive impacts lead to neurodegeneration later in life. It is also believed that the repetitive impacts and sub-concussive impacts cause injury to the tiny blood vessels in the brain. The damaged blood vessels, in turn, result in damage to the blood-brain barrier, a structure designed to protect the brain.

When the blood brain barrier is damaged by repetitive trauma, an abnormal immune mediated inflammatory response is triggered causing the production of neurochemicals. The neurochemicals and inflammatory response are normally protective after an initial head injury. However, a problem arises when the brain is subjected to subsequent repetitive sub-concussive impacts before the protective neurochemicals and inflammatory changes, from the initial head injury, have had time to return to normal. The subsequent, repetitive head injuries result in an abnormal and harmful overproduction of neurochemicals, along with an exaggerated inflammatory response, which proves detrimental rather than protective to the brain. This harmful response damages the brain tissue and eventually leads to the irreversible death of brain cells. Over time, this abnormal inflammatory pathway triggered by repeated head injuries (especially those encountered in sports) eventually leads to changes in a brain protein called tau.

The tau protein, important to cognitive brain cells, typically fosters stability, facilitating efficient communication among these cells to support normal thinking and behavior. Once the tau protein becomes compromised, its inability to stabilize brain cells leads to a decline in their efficiency and effectiveness. The spread of damaged tau protein across the brain results in the progressive loss of essential cells responsible for cognition, emotion regulation, and behavior control, manifesting increasingly noticeable symptoms of cognitive impairment and behavioral changes.

Indirect impacts on a person's head, whether small or large, are capable of creating shear forces that have the potential of injuring the brain in several ways. As will be discussed in more detail below, the shear forces cause the brain to rotate within the cranial cavity. The rotation of the brain is capable of causing 1) compressive brain injuries, expansive brain injuries, and torsional brain injuries, all of which are potentially destructive to the brain blood barrier. Additionally, the shear force impacts and their rotational shear forces on the brain have been shown to damage the micro-vasculature, deep inside the brain, that supplies the brain neurons. When these neurons are damaged at the cellular level the result is CTE.

These types of injuries are particularly troubling when one considers that athletes often begin playing sports as early as six (6) years of age. When an athlete or player receives a direct hit to the head, the brain can move up to +/−1mm in a linear plane. Usually, a high-force impact creates brain swelling and detectable concussion symptoms. Alternatively, when an athlete or player receives a lower force angled blow/impact that causes the player's head to rotate, the brain can rotate up to +/−3-5mm on a rotational plane inside the skull.

What is unknown at this time and which this invention also addresses is individuals in other occupations or activities that expose them to smaller repetitive head impacts. The following are examples of others that may be exposed to repetitive head impacts, which may increase their potential of developing CTE later in life.
Construction workers often experience head impacts on the structures they are building.
Mechanics often work under vehicles or in tight spaces.
Firefighters or EMT personnel often work in unstable structures that can fall on them.
Warehouse workers are exposed to items falling from high spaces.

Further, because the brain can move more rotationally around a vertical axis of the body, than directly back and forth in an oscillating movement, injuries can occur at the cellular level from a lower rotational force than from a direct force on the head. The risk of long-term CTE increases with cumulative rotational forces at a level smaller than what causes acute concussions. This new knowledge has unveiled a number of shortcomings in the current identification, detection, treatment, and prevention of brain injuries from concussions. In particular, there is a need to not only protect against and detect larger impact forces that can cause an acute concussion, but to also protect against and detect the smaller and more frequent sub-concussion and/or rotational forces than can result in longer-term CTE and its associated dementia-like symptoms.

As mentioned above, anyone capable of encountering head trauma or rotational head movement is susceptible, including but not limited to military soldiers, industrial workers, firefighters, police officers, self-defense instructors, and many more that will likely be identified as research continues. Anyone in any environment where a head impact or contact is possibly at risk of sub-concussive and/or rotational brain injuries.

The above is not intended to limit the scope of the invention, or describe each embodiment, aspect, implementation, feature, or advantage of the invention. The detailed technology and preferred embodiments for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

Certain embodiments of the present disclosure are illustrated, by way of example, in the figures of the accompanying drawings, in which like references indicate similar elements.

FIG. 1A is an illustration of a direct impact to a user's head.

FIG. 1B is an illustration of a rotational impact to a user's head illustrating zones of deep level brain injuries.

FIG. 1C is an illustration of an impact detection device and its sensor locations with respect to user's head, in accordance with the embodiments of the invention.

FIG. 1D is a side view of different layers of a brain, in accordance with the embodiments of the invention.

FIG. 1E is a top view of a user's head with a head gear positioned thereon and illustrating rotational triangulation, in accordance with the embodiments of the present invention.

FIG. 2 is a flow diagram of an example of functionality of the impact detection device, in accordance with the embodiments of the invention.

FIG. 3 is a flow diagram of an example of functionality of the impact detection device, in accordance with the embodiments of the invention.

FIG. 4 is a perspective view of an inner surface of the impact detection device, in accordance with the embodiments of the invention.

FIG. 5 is a perspective view of an inner surface and back surface of the impact detection device, in accordance with the embodiments of the invention.

FIGS. 6 and 7 are flow diagrams of examples of functionality of the impact detection device, in accordance with the embodiments of the invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the scope of the invention as defined by the appended claims.

### DETAILED DESCRIPTION

The present invention outlines example embodiments of an impact and injury detection device or system that can be positioned on user's or subject's head, or another part of their body. The present invention can detect different types of impacts, including but not limited to direct and rotational impacts. It is also able to locate zones of potential injuries based upon the location and force of the detected impact(s) and to provide suggested courses of action, including but not limited to resting and seeking medical care. The present invention is also able to detect, monitor, and protect against impact forces, including but not limited to sub-concussions, acute concussions, and rotational impact forces.

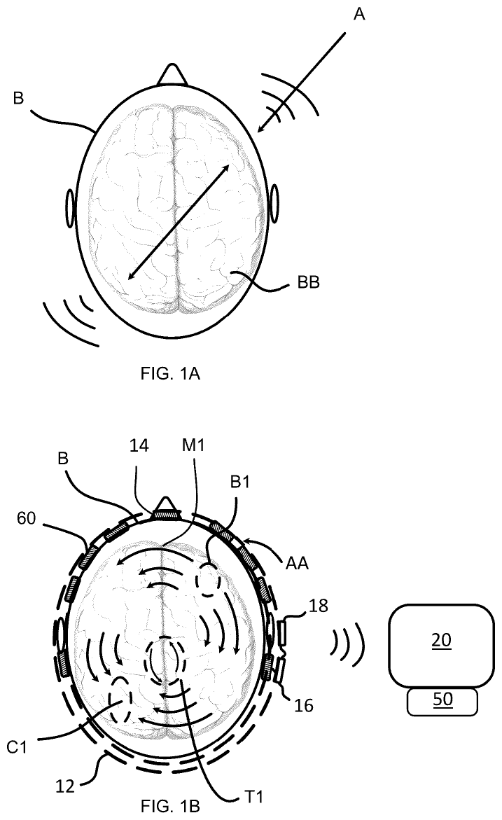

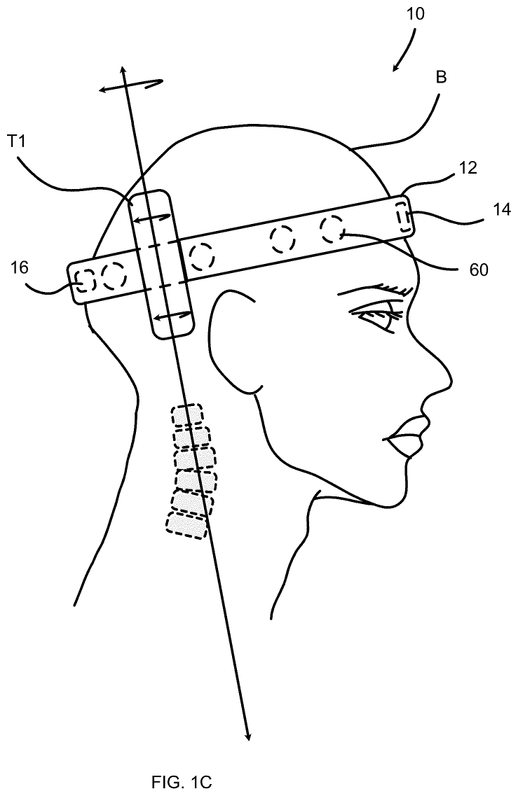

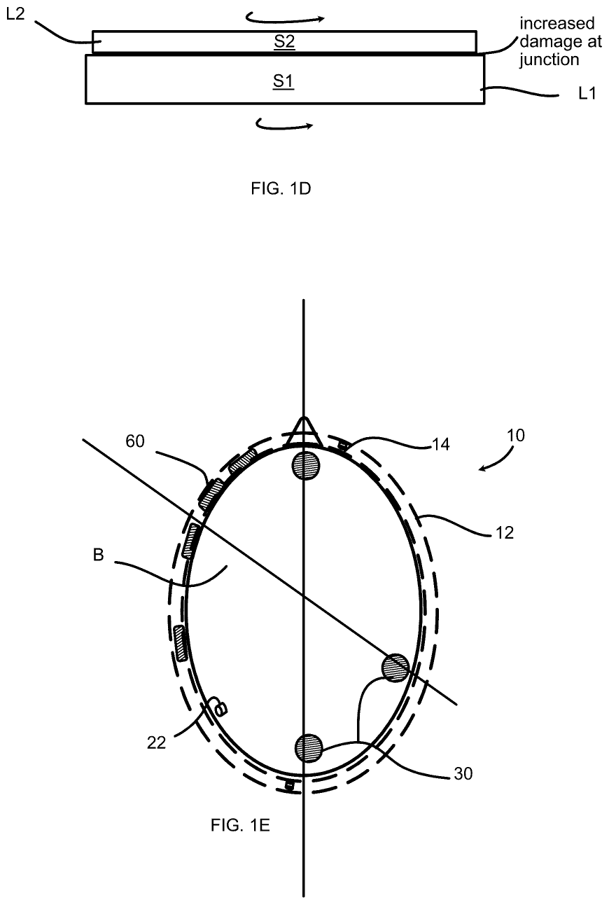

The present invention, as illustrated in FIGS. 1-7, comprises an impact detection system 10 having one or more sensors 14 that can detect points and angles of impact, absolute acceleration or force on the head, or the rotational forces of different parts or anatomical structures of a subject's head and brain. The configuration of system 10 of the present invention enables it to detect sub-concussions, acute concussions, and rotational injuries, including but not limited to compressive injuries, expansive injuries, torsional injuries.

The system 10 of the present invention is also able to accurately measure rotational forces, identify zones of injury, and provide a risk assessment and risk trending of the potential injuries to the athlete or subject, and the possibility of the athlete, player or subject later developing CTE. In this manner, system 10 of the present invention can provide an assessment or range from a point of being clinically normal to the start of display symptoms, and through the continued progression of the disease.

As illustrated in FIG. 1A, most conventional impact detection devices are only able to detect a location of impact A on athlete's or subject's head B. These conventional devices use only force to determine if there was a concussive event. As a result, only direct impacts, as illustrated in FIG. 1A, were concussive events because they generated forces high enough to reach a measurement that is within the criteria for a concussive event (e.g., 10 g).

The impact detection system 10 of the present invention, as illustrated in FIG. 1B, includes a substrate or headgear 12, configured to be placed about an athlete or user's head B. The substrate or headgear 12 may comprise a headband, helmet, or other head covering garment that extends at least partially about the subject's head. Headgear 12 includes one or more sensors 14 mounted to or in a portion of headgear 12. Sensors 14 are configured to measure one or more states, or a change in state, being experienced by the user's or subject's head B or brain, including but not limited to acceleration, force, compression, temperature, and the like. Sensor 14 can also be multifunctional, detecting more than one state, or more than one change in state.

Substrate or headgear 12 can include any number of sensors 14. In one example embodiment, substrate, or headgear 12 includes at least four sensors 14. In another example embodiment, substrate, or headgear 12 includes less than or more than four sensors 14. The number and location of sensors 14 can improve accuracy of rotational measurements and suitability to different sport/environments. Sensors 14 can be located on substrate or headgear 12 proximate to the one or more of a user's or subject's frontal, temporal, or occipital bones. Any location on the head or body that may experience an impact may have a sensor 14 positioned near it to detect and measure the parameters discussed herein.

As illustrated in FIG. 1B, when an indirect impact AA occurs, sensors 14 of the present invention can detect the location of the indirect impact AA and the force of the indirect impact AA. A communication assembly or computing device 16 connected to, in communication with, or disposed in substrate 12 is in operational communication with sensors 14. Communication assembly 16 includes an antenna 18 that communicates with a data processor 20, that can be remote from the substrate or headgear 12.

When an impact occurs sensor(s) 14 initially measures an absolute force measurement to detect a risk of an acute concussion, sub-concussion, or brain injury. Next, sensor 14 performs a measurement or cumulative measurements to detect a measure of rotational forces to the brain, which assesses CTE risk and probability. These measures can trigger an alarm - either in real-time during the activity, such as while playing a sport, or during a break, such as when the user is on the sidelines. When the forces on the head B, and propagated to the brain BB, have exceeded the limits for either a pre-defined impact tolerance, a concussion or a sub-concussion causing CTE, the user or player can be protected against acute and long-term brain injury. Protection includes removal from the activity or game, use of specialized headgear that reduces impact forces, including rotational forces, and regular monitoring to name a few examples that are within the spirit and scope of the invention.

As particularly illustrated in FIG. 1B, the mechanism of action of CTE has implications for assessing long-term risk of developing clinical dementia symptoms. As a subject's head is impacted by an indirect hit, subject's head B strongly rotates (illustrated as rotating to the left). The impact and strong rotation create several injury points, locations, or zones B1, C1, and T1. The initial impact creates an expansion zone or injury B1 at or near the site of the impact AA. The location of the indirect impact AA causes the expansion zone B1 of the brain BB to expand, resulting in bruising and tears of brain tissue, nerves, and vascular structures. As the subject's head B rotates the force wave radiates around the subject's brain converging into a compression zone or injury C1, that is approximately located opposite of the expansion zone B1. The compressive forces cause the brain tissue and cells to collide, again causing an injury to brain tissue, nerves, and vascular structures of the subject's brain BB.

While the force wave from the impact radiates through the subject's head B, the subject's head B continues to rotate about a user's vertical axis formed by the subject's spine. The strong rotational movement causes the subject's brain BB to also rotate inside of the subject's skull, resulting in the formation of a torsional zone or injury T1. As illustrated in FIGS. 1B and 1C, torsional zone or injury T1 is located slightly posterior to a central portion of the subject's head B.

The rotational movement of the subject's brain causes sheering forces between the brain and blood brain barrier (“BBB”). The twisting of the brain causes injury to the white matter deep in the brain and along the mid-line M1 of the subject's brain.

As illustrated in FIG. 1D, the rotation forces of the subject's brain can cause different layers (e.g., white matter (L1) and the BBB (L2) to rotate at different speeds or accelerations (S1 and S2). For instance, the mass of the white matter L1 rotates at a lower speed or acceleration than the BBB L2. This difference in their rotational speed or acceleration also results in tearing and damage at the cellular level.

System 10 of the present invention can also include one or more parameter sensors 30 that are connected to the substrate 12 and/or positioned somewhere on a subject's body or head B. The parameter sensors 30 can comprise a sensor incorporated into substrate 12 that is configured to measure the circumference of the subject's head B. Parameter sensors 30 can also be configured to identify the location of the subject head B. For instance, a parameter sensor 30 can be positioned proximate to a subject's forehead while another parameter sensor 30 is positioned proximate to the back of a subject's head B. A third parameter sensor 30 can be positioned on another location of a subject's head B, such as for example, a subject's ear. The parameter sensors 30 can work individually or together to triangulate or locate a subject's head in space and to transmit this location data to the processor 20.

As illustrated in FIG. 1E, substrate or headgear 12 can also include one or more accelerometers 32 that are configured to accurately calculate the rotational, peak acceleration and deceleration the subject's head experiences. In one embodiment of the present invention, one or more accelerators 32, such as MEMS accelerators, is positioned on a subject's head B to detect both peak rotational acceleration and peak linear acceleration. Because acceleration is linearly related to force, the acceleration data is proportional to force. The rotational acceleration data over a threshold level is then integrated over time to provide a measure of the cumulative rotational forces over a period of time. While some example sensors are described herein, it should be understood that the sensors may also comprise temperature sensors, gyroscope sensors, humidity sensors, light sensors, touch sensors, tilt sensors, and the like.

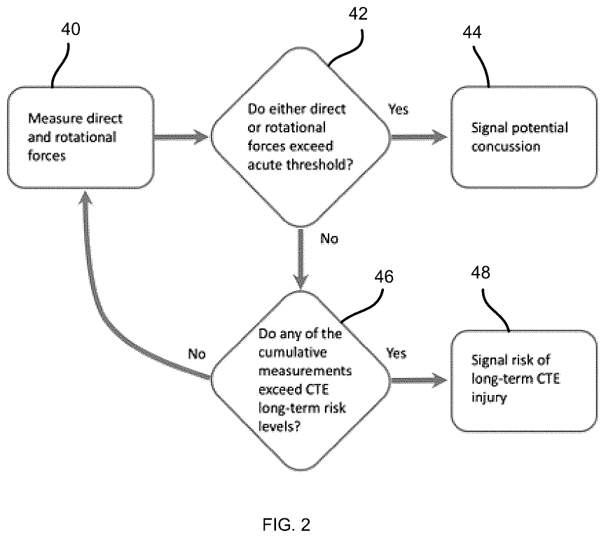

The system 10 of the prevention can detect the risk of long-term CTE injury. The system 10 includes a processor 20 having an algorithm or software that utilizes a decision matrix, an example of which is illustrated in the flow diagram of FIG. 2. The processor 20 and software work in conjunction with substrate or headband 12, sensors 14, parameter sensors 30, and accelerometers 30, individual or in combination, to determine relative anatomical locations and to measure cumulative rotational forces over a time period: Upon an impact event, processor 20 communicates with sensors 14, parameter sensors 30, and accelerometers 32 to determine if there is an impact event and to determine the kind of impact event 40.

If there is an impact event, processor 20 determines if the forces (indirect or direct) experienced exceed an acute threshold 42. If a threshold is exceeded, processor 20 will notify the subject or one or more other individuals that an event, such as a head impact, concussion, sub-concussion may have occurred 44.

If processor 20 determines an threshold or level has not been exceeded at step 42, processor 20 then determines if any of the detected cumulative measurements exceed predefined CTE long-term risk levels 46. If CTE long term risk levels are exceeded, processor 20 signals the subject and/or one or more other individuals of the risk. If no impact, concussion, acute concussion, sub-concussion, or cumulative levels exceed predefined limits, system 10 can reset and continues monitoring for another impact event.

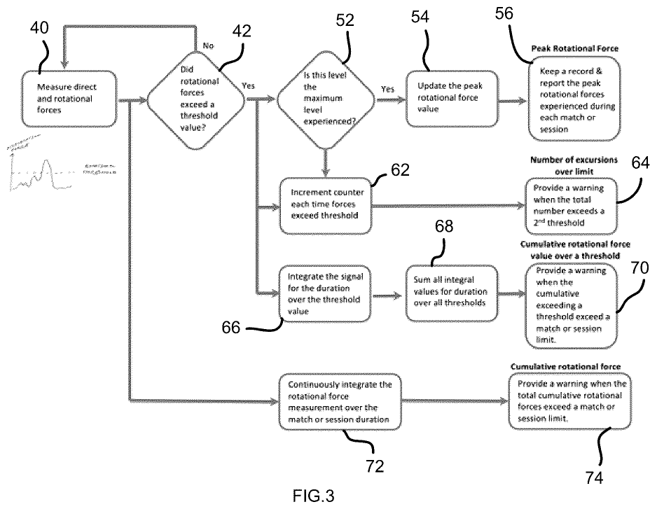

As illustrated in FIG. 3, system 10 can also perform multiple measurements that can be utilized and combined to create a risk measure for CTE and to assess brain injury. The measures include but are not limited to:
Peak rotational force: this identifies the peak rotational force over a time period and compares this to a threshold. This can be useful to determine whether a force occurred that could cause immediate and acute concussion and sub-concussion damage.
Cumulative number of rotational forces over a threshold level and period: this measure establishes a baseline for rotational force risk provides a count of the number of times during a time period that rotational forces exceed this threshold. This indicates the cumulative rotational forces extend into a danger level for damage at the cellular and microvascular level. It should be noted that this threshold level will be significantly lower than what might be indicative of a symptomatic concussion.
Cumulative forces over a threshold level: By integrating the forces experienced over the time period when they exceed the threshold level, a cumulative magnitude of dangerous rotational forces are calculated. Further the average rotational force for each hit and for the total time above a threshold can be calculated. Summing these integration figures together over a total time period would result in a figure of merit for total CTE risk and potential likelihood for clinical injury.

As illustrated in FIG. 3, system 10 can detect if an impact has occurred and measures the direct or rotational forces at step 40. The system 10 then determines if a threshold force value has been exceeded at step 42. Next, system 10 determines if the force level experienced/measured is a peak or exceeds prior levels 52. If the level is a peak, system 10 updates the peak level 54 and keeps a record and, optionally, reports a peak level experienced during each event 56.

If system 10 determines that an experienced level is not a peak, it records the level as an incremental impact level at step 62. The system 10 is then able to issue a warning if a predefined number or rotational forces is exceeded 64. System 10 can store the value of each rotational force experienced at step 66. System 10 is then able to calculate or sum the rotational forces experienced over a time period at step 68 and issue a warning if a cumulative or total rotational force experienced is exceeded at step 70. Similar to other processes performed by system 10, it continuously integrates the rotational force measurements over a match, a season, a career, or lifetime of a person, see step 72. Any period of time may be monitored. System 10 is able to provide a warning whenever total cumulative rotational forces exceed a level for any time period at step 74.

As discussed above, system 10 can monitor cumulative events that have happened over time. System 10 includes storage 50 in communication with processor 20, any or all of the sensors 14, 30, and 32, or any combination thereof. Separate storage 50 may be in communication with processor 20 and disposed on or in communication with substrate 12. The average rotational forces over a certain amount of time, such as an athletic event, where the processor 20 and software can provide different outputs, including an overall output for a defined period, e.g., game/season/return to play period. These cumulative outputs along with outputs from other activities and sporting events can be kept in storage or memory 50, where processor 20 is able to monitor cumulative impacts and forces experienced by a subject over a period of time, such as their entire life, during a professional career, or during a game.

The system 10 can also use storage 50 to monitor and store:
a subject's age, weight gender, head circumference, etc. ;
a number of impacts;
a type of impact (direct or indirect);
a magnitude of impacts;
a location of each impact; and
past medical diagnoses and past medical diagnoses associated with each impact and its location.
Amount of rest between significant rotational forces

In one configuration of the present invention, substrate, headgear, or other protective device 12 also includes one or more bioelectrical sensors 60 capable of detecting brain waves signals using an electroencephalograph (EEG). These bioelectrical sensors 60 and the signals they detect can be utilized to help discern acute concussion, sub-concussions, immediate injury, long-term CTE risk trending, and CTE progression. As illustrated in FIG. 1B, bioelectrical sensors 60 are generally positioned or mounted on an inner surface of headgear or head band 12 where the bioelectrical sensors 60 can come into contact with a subject's skin.

System 10 is able to conduct impact analysis with or without conducting or performing EEG analysis. In one example embodiment, system 10 conducts an impact analysis to calculate the amount and types of forces on a user's head and brain. The impact analysis can be used as an approximation of a level of brain injury. When used in conjunction with the EEG analysis (e.g., detecting brain signals over time) a direct link, comparison, or correlation can be made with the impact analysis. Computing device 16 is able to analyze the impact analysis and EEG analysis and generate a suggested action (e.g., remove subject from game or work, conduct head scan, etc.) to help prevent or reduce injury to a subject or user's brain.

The present invention is also able to output additional procedures, including turning on or initiating bioelectrical sensors 60 in the headgear or head band 12 to detect brain signals or waves to detect if a concussion has occurred. The present invention can also output a procedure separate from the capabilities of headgear or headband 12, such as the use of a functional near infrared (FNIR) system to locate and identify sub-cranial hemorrhage. While the FNIR is helpful after a potential injury has occurred, it is not able to be used proactively during play or sport. Based upon the readings from the headgear 12, either alone or in conjunction with readings from and separate diagnostic procedures; a decision can be made for a corrective or protective course of action, including, use of specialized headgear, determination of a date to return to the activity or sport, or administration of medication or therapy. The list of output decisions is an example and should not be considered limiting.

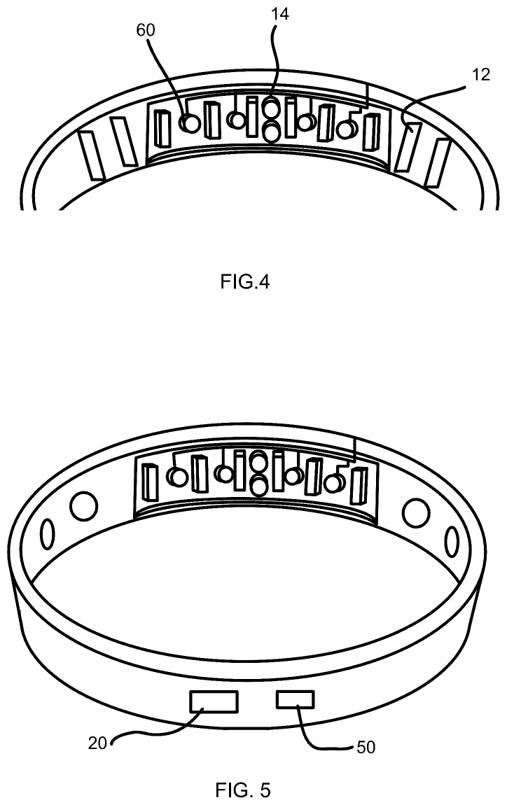

The headband 12 configuration of the present invention, shown in FIGS. 4 and 5, can be integrated to be used during athletic events, e.g., training

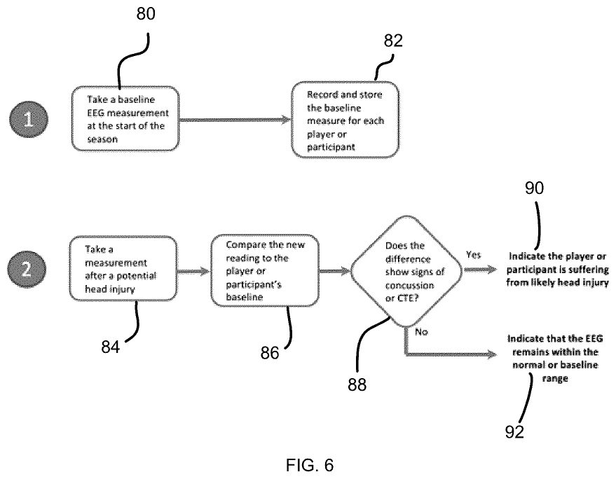

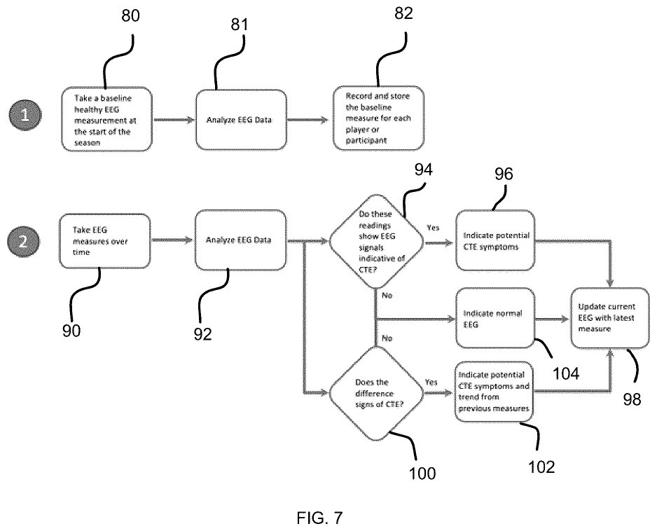

and game play. Further, the ease of its use allows baseline measures to be taken before the season or match and after a potential injury. Observing a differential measurement between the initial template and a subsequent data collection allows greater resolution than gross and common measures designed to be used only after injury occurs. Beyond an improved resolution, the system of the present invention accounts for individual user variation that is likely to occur from person to person. FIG. 6 illustrates a method for baseline data collection and evaluation. In an example embodiment of the present invention, system 10 can be used at the start of a time period (e.g., start of a season) to obtain a baseline EEG measurement 80. The baseline EEG measurement can be stored for each player or participant 82. Whenever an injury is detected or experienced, system 10 can initiate an EEG reading 84. System 10 is then able to compare new readings with the baseline reading (step 86) to determine if a change has occurred (step 88) and if the change amounts to a sub-concussion, concussion, or CTE. System 10 is then able to issue a warning (step 90) or indicate that there has been no change (step 92). System 10 can initiate an EEG reading at anytime and detection of an impact is not a requirement.

The difference measures could be from standard power frequency analysis, reaction to specific events to observe evoked potentials, or an artificial intelligence or machine learning algorithm designed to specifically pick out differences that indicate a concussive or other head injury.

In addition to showing acute concussion during the field use, the same headgear or wearable 12 could be utilized to indicate early onset of CTE clinical presentation and trend them during a time that is still clinically normal until the user begins to develop clinical symptoms. Current science indicates that CTE presents similarly to Alzheimer's Disease (AD) and Alzheimer's Disease Related Dementia (ADRD). Following damage at the cellular and microvascular level, plaques form in the brain of both AD/ADRD and CTE patients. Further CTE sufferers exhibit many of the same symptoms of AD/ADRD patients: memory loss, confusion, and executive function disability to name a few.

Alzheimer's disease is the most common form of dementia. According to the CDC, as many as 5.8 million Americans lived with Alzheimer's disease

in 2020. This number is projected to triple to 14 million people by the year 2060. Ask is the best-known risk factor of Alzheimer's with risk significantly increasing past the age of 60. Genetics may also play a role in the development of this disease. Common symptoms of Alzheimer's include memory loss that disrupts daily living, trouble handling financial responsibilities, difficulty completing familiar tasks and changes in mood, personality, or behavior. Research has suggested that physiological changes in the brain often occur even before symptoms become apparent. In fact, changes in the brain can begin to take place up to 20 years prior to the onset of first symptoms, however, screening methods that could detect pre-symptomatic changes such as MRI and EEG are rarely prophylactically applied.

Common treatments for Alzheimer's disease include a variation of pharmaceutical and lifestyle interventions. Generally, the goal is to help patients, or individuals genetically prone to disease development, maintain brain health to slow or delay symptoms. Physical health is highly correlated with disease severity. It is strongly recommended that patients vigorously treat hypertension while maintaining a regular exercise routine. Other recommended lifestyle interventions include maintaining social engagement, stopping smoking, managing hearing loss, depression, and obesity (Carillo 2017). Further, more involvement with intellectually stimulating activities such as verbal, spatial, and relational memory challenges are correlated with slower disease progression and improvement of cognitive functioning (Heneghan et al 2022). Chemically, Alzheimer's is characterized by two hallmark pathologies that are being addressed through pharmaceuticals: β-amyloid plaque deposition and neurofibrillary tangles of hyperphosphorylated tau (Weller et al 2018). These aliments are typically medicated by targeting amyloid β such as donepezil, galantamine, rivastigmine and memantine. Newer drugs are now targeting tau-targeting therapies since the tau protein seems to be more highly correlated with severity of cognitive decline (Vaz & Silvestre 2020).

Evidence suggests that these brain changes may be monitored through electrophysiology. Most of this analysis is targeted in areas of the brain that are known to be heavily correlated with higher levels of cognitive function. Processes such as the regulation of memory, planning and executive functioning can best be measured through frontal and temporal lobe analysis. Recent research using both EEG and fNIRS integration has found dynamic cortical connectivity alterations in Alzheimer's disease. Specifically, those with Alzheimer's display weaker connectivity to the orbitofrontal and parietal regions in high alpha band and beta band. Additionally, these brain networks are characterized by lower degree and clustering coefficient in the frontal regions (Li et al 2019). Moreso, EEG findings show slowing, decreased EEG synchronization and frontal shift of neuronal generators of fast frequencies (Smailovic & Jelic 2019; Ouchani et al 2021; Jeong 2004). These measures correlate with molecular and clinical imaging biomarkers of Alzheimer's, while also aiding in discriminating between AD and other forms of dementia (Dauwels et al 2010). Still, more large-scale longitudinal studies need to confirm these diagnostic criteria, while also considering measurements in more naturalistic environments (Perez-Valero et al 2022). Specifically, a contrast between working and resting state EEG recordings, varying wakefulness with sleep, would further allow more accurate representation of low-level changes (Vecchio 2013; Houmani et al 2018).

Targeting these similar mechanisms can also enable the early detection of CTE, potentially enabling the early identification of patients of all ages and slowing the progression of the disease. Advancements in brain-computer interfaces and EEG research have shown the ability to identify and assess a variety of these physiological issues. We aim to provide a platform that can collect cognitive insights virtually anywhere, anytime, creating the potential to shift the standard-of-care for identifying and monitoring the impacts of CTE. Our system is enabled by two core technologies: nanotechnology-based sensor electrodes and algorithms inspired by neural mechanisms. Coupling our clinical-grade EEG wearable with algorithms that detect brain changes due to CTE has the potential to change the understanding of CTE progression and help prevent or slow further life-debilitating injury.

FIG. 7 shows a method to measure and trend CTE prognosis from clinically normal to mild-severe symptoms. As discussed above, system 10 can take a baseline EEG measurement (step 80), analyze the EEG data (step 81), and then store or record the baseline measurement for each player or monitored individual (step 82). System 10 can take EEG readings over any time period (step 90) and analyze each EEG reading (individually or

cumulative) (step 92). System 10 can determine if the EEG data indicates CTE (step 94). If the EEG readings indicate CTE symptoms (step 96) it can update the current EEG measurements with the newest data (step 98) and issue a warning to the person being monitored, medical staff, and/or other individuals and systems.

System 10 can detect if there are different signs of CTE (step 100) and either indicate a finding of potential CTE symptoms and trend from previous measures (102). System 10 can then update current EEG data with the current EEG data and/or issue a warning to the person being monitored, medical staff, and/or other individuals and systems. If no CTE symptoms are indicated system 10 can indicate a normal EEG reading (step 104).

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. A method of detecting a change in brain state to a user's head having one or more sensors positioned on a headgear worn by the user, the method comprising the steps:
detecting, by a first sensor located on the headgear, a first location of the first sensor in space;
detecting, by a second sensor located on the headgear, a force of an impact to the second sensor;
detecting, by the first sensor, a second location of the first sensor in space;
in response to detecting the force of the impact, generating, by a computing device, a change in movement of the first sensor from the first location to the second location;
identifying, by the computing device, a rotational acceleration of the first sensor from the first location to the second location;
performing an impact analysis, by the computing device, comparing the force of the impact detected by the second sensor and the rotational speed of the first sensor to a threshold level data; and
performing an EEG analysis, by the computing device, comparing detected brain signals to the baseline level data;
wherein a deviation from the baseline level data or threshold level data correlates to a change in state of the user's brain.

2. The method of claim 1, further comprising detecting, by the second sensor located, a subsequent force impact.

3. The method of claim 2, wherein in response to the subsequent force impact to the second sensor, further performing a subsequent impact analysis, by the computing device, comparing the first impact to the subsequent impact, wherein the subsequent impact analysis is compared to at least one EEG analysis.

4. The method of claim 1, further comprising the step of determining, by the computing device, a rotational force based upon the force impact on the second sensor and the rotational speed of the first sensor, wherein the rotational force is an approximation of the rotational force of the user's head.

5. The method of claim 4, further comprising the step of detecting, by the first sensor and the second sensor, a subsequent rotational force, further performing a subsequent impact analysis, by the computing device, comparing the rotational force to a subsequent rotational force, wherein the subsequent impact analysis is compared to at least one EEG analysis.

6. The method of claim 1, wherein the first sensor comprises a gyroscope and the second sensor comprises an accelerometer.

7. The method of claim 1, wherein at least the first sensor is supported on a portion of a headgear that is located proximate to a forehead of a user.

8. The method of claim 4, wherein the headgear comprises a protective headband.

9. The method of claim 1, further comprising the step of storing, by the computing device, the force of the impact to the second sensor and the rotational speed of the first sensor.

10. A brain state monitoring system worn about a user's head, the system comprising:
a headgear extendable about a portion of the user's head, the headgear having an inner side surface;
at least one force sensor incorporated into a portion of the headgear;
at least one parameter sensor positioned on a portion of the headgear;
a computing device having a processor in operative communication with the at least one force sensor and the at least one parameter sensor, the computing device processor is configured to:
receive a first location of the at least one parameter sensor in space;
receive a force impact detected by the at least one force sensor;
receive a second location of the at least one parameter sensor in space after receiving the force impact from the at least one force sensor;
determine a rotational acceleration by comparing the first location and the second location of the at least one parameter sensor;
perform an impact analysis by comparing the force of the impact to the first sensor and the rotational acceleration of the parameter sensor to a threshold level data; and
perform an EEG analysis by comparing EEG signals prior to a force impact and after a force impact;
wherein a deviation from baseline EEG data or the threshold level data correlates to a change in brain state of the user.

11. The system of claim 10, wherein the computing device is further configured to determine a rotational force based upon the force impact on the at least one force sensor and the rotational speed of the at least one parameter sensor, wherein the rotational force correlates to a CTE event, a concussive event, or a sub-concussive event.

12. The system of claim 10, wherein the computing device is further configured to perform a subsequent impact analysis or EEG analysis upon detection of a subsequent force impact to the at least one force sensor.

13. The method of claim 11, wherein the computing device is further configured to perform subsequent impact analysis or EEG analysis upon detection of a subsequent rotational force detected by the at least one force sensor and the at least one parameter sensor, wherein the rotational force and subsequent rotational force are an approximation of cumulative rotational forces on the user's head.

14. The system of claim 10, wherein the at least one parameter sensor comprises a gyroscope sensor.

15. The method of claim 10, wherein the force sensor is positioned proximate to a forehead of a user.

16. The method of claim 15, wherein the headgear comprises a protective headband.

17. The system of claim 10, further comprising a storage configured to receive and store data, wherein the storage is in operative communication with the computing device.

18. The system of claim 10, further comprising a remote display in operative communication with the computing device, wherein remote users are able to monitor impact events and EEG analysis of the user wearing the headgear.

19. The system of claim 18, further comprising a remote computing device in operative communication with a number of different users each wearing the system, wherein a remote user is able to monitor a number of different users.

20. The system of claim 10, wherein the computing device is further configured to recommend an action based upon the impact analysis or EEG analysis.
